(19) United States
(12) Patent Application Publication
(10) Pub. No.: US 2007/0292561 A1
(43) Pub. Date: Dec. 20, 2007

(54) Ami-Deficient Streptococcus Thermophilus Strains With Reduced Post-Acidification

(76) Inventors: Peggy Garault, Montlhery (FR); Jean-Michel Faurie, Jouy En Josas (FR); Jerome Mengaud, Saint Martin En Briere (FR); Anne Druesne, Bacle (FR); Gaelle Quere-Chassang, Villebon Sur Yvette (FR)

Correspondence Address: FOLEY AND LARDNER LLP;SUITE 500, 3000 K STREET NW, WASHINGTON, DC, 20007, US

(21) Appl. No.: 11/577,622

(22) PCT Filed: Oct. 21, 2005
(86) PCT No.: PCT/EP05/55443

§ 371 Date: May 24, 2007

(30) Foreign Application Priority Data
Oct. 22, 2004 (FR) 0411275

(51) Int. Cl. A23C 9/123; A23C 9/127; A23L 1/105; C12N 1/20

(52) U.S. Cl. 426/18; 426/43; 426/61; 435/253.4

(57) ABSTRACT

The invention relates to Ami-deficient wild-type strains of Streptococcus thermophilus with reduced post-acidification. The invention also relates to the use of said strains for the preparation of fermented food products and to the fermented food products thus prepared.

This invention relates to the field of lactic bacteria used in the food industry.

More precisely, the invention relates to natural strains of Streptococcus thermophilus or natural variants of such strains, said strains or variants being AMI-deficient with reduced post-acidification.

The invention also relates to the use of said strains for the preparation of fermented food products and to the fermented food products thus prepared.

The choice of lactic bacteria for the production of food products, particularly fermented dairy products, requires a number of criteria including acidifying activity and the formation of aromatic compounds responsible for the product's organoleptic properties, as well as the production of thickening agents which play a role in texture and creaminess.

Acidifying activity is essentially characterised by three parameters: (i) the kinetics of acidification; (ii) titratable acidity or final fermentation pH which affects the organoleptic properties of the product and its suitability for storage; and (iii) post-acidification which develops during storage of the product.

A high acidification rate makes it possible to reduce the period during which the preparation is sensitive to contaminants and thus reduces the risk of bacterial contamination.

Increasing the rate of acidification also improves the economics of the process by increasing the productivity and flexibility of the industrial material.

The post-acidification properties of the strain are particularly important for the storage of products. Fresh fermented products are generally stored at temperatures between 4° C. and 8° C. for a period of 4 weeks. While the metabolic activity of bacteria is reduced by cold storage, it is nonetheless not blocked. This remnant activity leads the production of lactic acid from lactose and results in a decrease pH and an increase in acidic flavour which degrades the organoleptic properties of the product.

Moreover, in addition to the criteria retained for their contribution to the product's quality, other more specifically linked elements are involved in the choice of strains, for example the fermentation temperature, acidification rate and resistance to phages.

Resistance to phages is a highly important criterion in the choice of strains in order to reduce the risk of phagic events during production since these are likely to block the entire production process for varying periods of time while decontamination is carried out.

French patent FR 2 725 212 describes a strain of S. thermophilus deposited at the National Collection of Microorganism Cultures (CNCM, Pasteur Institute, Paris, France) under number I-1477 which has rapid acidification kinetics allowing a high degree of acidity and which does not post-acidify in the course of storage of fresh fermented dairy products.

Another S. Thermophilus (registered on 30 Dec. 1994 at CNCM under number I-1520) is described in French patent FR 2 771 600 as having a reduced post-acidification capacity.

Nonetheless, is not advantageous to use such strains to manufacture functional foodstuff (i.e. foodstuff which has a beneficial effect on one or more target functions in the organism), especially those containing the protein and/or peptide-based bioactive food ingredients of interest. These strains remain capable of metabolising (in other words, of breaking down for use as sources of nitrogen) proteins and/or peptides including the functional proteins and/or peptides of interest which we evidently want to preserve.

This is why it is important for the food industry to have available fermenting bacteria that: (i) have excellent viability; (ii) very low or practically nonexistent post-acidifying activity; and (iii) a very low capacity for metabolising proteins and/or peptides.

The food industry also has to take into consideration a fourth constraint when developing products for human and/or animal food into which microorganism are incorporated, more particularly live microorganisms. Genetically modified (GMOs or mutants) organisms (in this case, microorganisms) are generally regarded with caution and apprehension by consumers. The negative image from which GMOs generally suffer means the public tends to boycott food containing GMOs. Therefore, given that consumers always want more and more transparency with regard to the contents of food products on offer and the origin of ingredients in these products, manufacturers prefer to offer GMO free products. it is therefore essential that industrially produced foodstuff containing microorganism are prepared using only natural strains or natural variants of natural strains.

It is precisely the above-mentioned set of constraints this invention responds by proposing natural strains of S. thermophilus or natural variants of such strains, said strains or variants being AMI-deficient with reduced post-acidification.

The growth of S. thermophilus in milk can be broken down into three growth phases:
a first exponential growth phase which does not depend on milk caseins;
a second so-called transitional growth phase; and
a third exponential growth phase, doubtless limited by peptide transport (Letort et al., 2002).

The oligopeptide transport system in S. thermophilus is an ABC transporter (ATP-Binding Cassette) consisting of a translocon of four proteins (AmiC, AmiD, AmiE and AmiF) and three highly similar binding proteins (AmiA1, AmiA2 and AmiA3). The growth of mutants in milk in which the oligopeptide transport system is altered is not optimal which demonstrates the importance of this transport system (Garault et al., 2002).

Within the scope of this invention, the Applicant has for the first time found natural strains and natural variants of S. thermophilus strains in which the oligopeptide transport system is at least partially altered. The Applicant has also shown that such strains and variants have particularly interesting milk acidification properties.

On the one hand, the pH becomes stable at the end of fermentation for AMI-deficient variants. In fact, the first growth phase in milk is not affected and these variants can retain a relatively rapid first acidification phase at the same time as having a much slower second acidification phase. They can present an acidification graph that is much slower at a higher pH than that at which acidification by an AMT-functional wild strain is slowed down. This “acidification plateau”, at the fermentation temperature, is of interest in that it allows the fermented mass to be stored at the fermentation temperature for much longer and this without substantial acidification. The de-clotting times for fermentation vats are fairly long given their size. The use of AMT-deficient variants thus makes it possible to limit pH changes in the fermented milk in the course of decantation (de-clotting with cooling down or direct packaging).

On the other hand, the pH threshold at which acidification is greatly slowed down depends on the amount of free amino acids and di-/tripeptides that can be transported by the AMI system and present in milk. It is therefore possible to steer the pH in order to stop fermentation by modifying the peptide content (qualitative and quantitative) of milk.

This invention therefore covers a natural strain of AMI-deficient S. Thermophilus with reduced post-acidification and its natural AMT-deficient variants having similar milk acidification characteristics, as well as their biologically pure cultures and culture fractions.

In the context of this invention, “AMI-deficient natural strain” or “AMI-deficient natural variant” refers to a strain or variant whose growth is stimulated by a mixture of free amino acids and not by a mixture made up solely of oligopeptides.

The term “natural AMI-deficient variants having similar milk acidification characteristics” refers to strains obtained by genetic modification starting with a reference natural strain, with the resulting strains having reduced post-acidification in the same way as the reference strain. In this context, the terms “variant” and “natural variant” are used to designate a strain obtained principally by mutation and selection of the reference strain whereas the term “mutant” is more specifically used to designate a strain obtained by directed mutagenic techniques, namely by genetic transformation using vectors applied to the reference strain.

According to one embodiment, the natural strain and/or natural variant is resistant to a toxic oligopeptide analogue transported by the AMI oligopeptide transport system.

In particular, said toxic analogue can be chosen from amongst aminopterine, triomithine and trilysine. This analogue is preferably aminopterine.

The term “resistance to aminopterine” is defined as observation, in the presence of a paper disc soaked in 200 μg of aminopterine, of an inhibition diameter less than or equal to about 2 cm, preferably less than or equal to about 1.8 cm, in a dish seeded with 100 μl of saturated bacterial suspension.

Such a definition will be extended with the necessary changes by the man skilled in the art to other oligopeptide toxic analogues transported by the AMI system in light of his general knowledge. Thus the man skilled in the art would also be able to vary the maximum values for inhibition diameters in an appropriate manner and as a function of the toxic analogues in question.

Preferably, the strain according to this invention is chosen from among:
the strain registered with CNCM on 10 May 2004 under number I-3211;
the strain registered with CNCM on Sep. 16, 2004 under number I-3301;
the strain registered with CNCM on Sep. 16, 2004 under number I-3302;
the strain registered with CNCM on Jan. 24, 2002 under number I-2774.

As an example of the acidifying properties of interest in the context of this invention and as demonstrated by the following examples, we point out that AMI-deficient strain I-3302 (variant of the mother strain AMI-functional I-3299 registered with CNCM on Sep. 16, 2004) makes it possible to obtain a pH variation of 0.29 between the pH at the end of incubation and the product's pH at the end of shelf life whereas the AMI-functional mother strain I-3299 gives rise to a pH variation of 0.56 between these two times. In this case, the advantage of using an AMI-deficient strain rather than an AMI-functional strain therefore resides in the 0.27 reduction in pH variation between the end of incubation and the end of the product's shelf life.

As mentioned above, AMI-deficient variants are obtained from a reference mother strain. Thus within the scope of this invention, this refers to natural variants obtained for example through mutation and selection as a function of acidifying properties. The techniques for this are known, as are the tests allowing acidifying properties to be regulated (namely Spinnier H. E., Corrieu G., 1989).

Contrary to strains I-3211, I-3301 and I-3302, which are natural AMI-deficient variants of AMI-functional mother strains (refer to experimental section below), strain I-2774 has been isolated and identified by the Applicant as being naturally AMI-deficient.

The strains according to the invention are of particular interest in the preparation of fermented food products.

Preferably, said fermented food products are dairy or vegetable products.

According to this invention the term “dairy product” refers to, in addition to milk, products derived from milk such as cream, ice cream, butter, cheese and yoghurt, as well as secondary products such as lactoserum and casein and any prepared food containing milk or milk constituents as the main ingredient (for example formula milk).

The term “vegetable product” refers to, amongst others, products obtained from a vegetable base such as fruit juices and vegetable juices including soya juice, oat juice and rice juice.

Moreover, the above-mentioned definitions for dairy products and vegetable products each cover any product based on a mixture of dairy and vegetable products, such as a milk and fruit juice mixture for example.

Thus, this invention also relates to a process for the preparation of a fermented food product in which a substrate is fermented with at least one live strain such as that described above.

It is advantageous in this process to contemplate the use of one or more bacterial strains such as those described above, mainly with at least one other live bacterial strain.

Advantageously, said other bacterial strain is a lactic bacteria strain such as Streptococcus spp.; Lactobacillus spp., namely L. bulgaricus, L. acidophilus and L. casei; Lactococcus spp. and Bifidobacterium spp.

Preferably, the substrate to be used in the above-mentioned process is chosen from among dairy substrates, namely milk or substrates based on vegetable raw ingredients such as fruits, cereals and soya. Therefore, the following can be used:
if the substrate is a dairy one, natural or reconstituted milk, skimmed or not, or milk based mediums or products of dairy origins; and
if the substrate is based on vegetable raw ingredients, fruit juices and/or vegetable juices.

The substrate can also advantageously include currently used elements in the food processing industry for the preparation of fermented products, namely milk based desserts, particularly solid elements such as fruits, oats or cereals for example, but also other liquid sweetened or chocolate-containing products.

This invention also relates to fermented food products whose organoleptic properties are preserved during storage. The term “organoleptic properties are preserved during storage” signifies organoleptic properties of the product that do not change (or not significantly for the consumer) in the course of time, at least until the products' expiry date (ED) and advantageously beyond that date.

According to one embodiment, said products contain at least one live strain in accordance with the invention.

According to another embodiment, the fermented food products according to the invention are likely to be obtained by means of the process described above.

Preferably, the fermented food products according to the invention contain at least one bioactive ingredient (or functional food ingredient), chosen in particular from among the proteins, peptides and analogues or derivatives of these.

The term “bioactive or functional food ingredient” refers to an ingredient that advantageously affects one or more target functions in the organism, independently of its nutritional effects. Thus the effect of such an ingredient might be to improve health and/or well-being and/or reduce the risk of occurrence of disease in the consumer who ingests normal quantities of said ingredient.

This definition can be extended to a foodstuff or product containing such an ingredient, said foodstuff or product henceforth itself becoming bioactive or functional.

The term “analogue” refers to any modified version of an initial compound, in this case a protein or peptide, said modified version possibly being natural or synthetic in which one or more atoms, such as the carbon, hydrogen, oxygen atoms or heteroatoms such as nitrogen, sulphur or a halogen have been added or eliminated from the compound's initial structure so as to obtain a new molecular compound.

A “derivative” in the context of the invention is any compound which has a similarity or structural motif in common with the reference compound (protein or peptide). This definition also covers, on the one hand, compounds which alone or in conjunction with other compounds can be precursors or intermediate products in the synthesis of a reference compound with one or more chemical reactions and, on the other hand, compounds which can be formed from said reference compound, alone or with other compounds, via one or more chemical reactions.

The term “derivatives” therefore covers at least hydrolysates, namely trypsic, of proteins and/or peptides and hydrolysate fractions, as well as mixtures of hydrolysates and/or hydrolysate fractions.

Among the bioactive food ingredients likely to be used in fermented food products according to the invention, the following can be cited as nonlimiting examples: peptide 91-100 of casein αₛ₁ (see European patent EP 0 714 910), peptide C6-αₛ₁194-199 (see American U.S. Pat. No. 6,514,941), peptide C7-β177-183 (see American U.S. Pat. No. 6,514,941), peptide C12-αₛ₁23-34 (see American U.S. Pat. No. 6,514,941), caseinophosphopeptides, α-casomorphin, casein α exorphin, casokinin, β-casomorphin, caseinomacropeptides (CMP) and the glycomacropeptides (GMP), casoxin, casoplatellins, fragments 50-53, β-lactorphins, lactoferroxin, the peptides Val-Pro-Pro (see European patent EP 0 583 074), Lys-Val-Leu-Pro-Val-Pro-Gln (see application EP 0 737 690), Tyr-Lys-Val-Pro-Gln-Leu (see application EP 0 737 690), Tyr-Pro (see application EP 1 302 207 and patent EP 0 821 968), Ile-Pro-Pro (see Nakamura et al., 1995 and Japanese patent JP 6 197 786), fragments, analogues, derivatives of these, proteins and/or peptides containing them and combinations thereof (for a review, refer to Danone World Newsletter number 17 dated September 1998).

The table 1 below lists the main functional peptides released by the hydrolysis of human and cow milk proteins.

TABLE 1

| | Functional | Milk | | |
|---|---|---|---|---|
| Original proteins | peptides* | source** | | Activities described |
| - α casein | α casomorphin | | C | opiate activity |
| | casein α exorphin | | C | opiate activity |
| | casokinin | | C | antihypertensive activity |
| - β casein | β casomorphin | H | C | opiate activity |
| | casokinin | H | C | immunomodulating activity + |
| | | | | antihypertensive activity |
| | CPP | H | C | effect on minerals |
| - κ casein | CMP = GMP | | C | modulation of gastrointestinal |
| | | | | motricity and release of |
| | | | | digestive hormones |
| | casozin | H | C | opiate antagonist |
| | casoplatellins | | | antithrombotic activity |
| - α lactalbumin | fragments 50-53 | H | C | opiate activity |
| - β lactoglobulin | β lactorphins | | C | opiate activity + |
| | | | | antihypertensive activity |
| lactoferrin | lactoferroxin | H | C | opiate antagonist |
| lactotransferrin | | | | |

*the amino acid sequences are note exactly the same.
**H: human milk - C: cow's milk

Table 2 below lists the principal physiological activities of functional peptides originating from milk and known to date.

TABLE 2

| Activities | Peptides | In vitro | In vivo animal | In vivo human | Ref. |
|---|---|---|---|---|---|
| Effect on | Caseinomorphin | Production of CCK | | | Beucher |
| digestion | (CMP) | by rat intestinal cells | | | 1994 |
| | | | Calf: after ingestion | Humans: after | Yvon 1994 |
| | | | of CMP (210 mg/kg), | ingestion of | |
| | | | inhibition of gastric | CMP (4 g), | |
| | | | secretion and | reduction in acid | |
| | | | reduction in plasma | secretion | |
| | | | CKK concentration | | |
| | β casomorphins | | Rabbit: after | | Ben Mansour |
| | | | introduction into the | | 1988 |
| | | | lumen, antisecretory | | |
| | | | effect on the ileum. | | |
| | | | Dog: after intragastric | | Schusdziarra |
| | | | administration, | | 1983 |
| | | | modulation of post-prandial | | |
| | | | insulinaemia; cancellation | | |
| | | | of this effect by naloxone | | |
| | Natural β | Several effects on | | | Tomé 1987, |
| | casomorphins | rabbit ileum | | | 1988 - Mahé |
| | and some of their | | | | 1989 |
| | analogues | | | | |
| | Non-metabolised | Stimulation of | | | Ben Mansour |
| | analogues of β | intestinal absorption | | | 1988 |
| | casomophins | of electrolytes | | | |
| | Casein | | Dog: administration of | | Delfillipi |
| | | | 10 g of casein/300 | | 1995 |
| | | | mL water by intragastric | | |
| | | | catheter: inhibition of | | |
| | | | large intestine motility, | | |
| | | | cancelled by naloxone | | |
| | | | vs 10 g of soya | | |
| | | | protein: no effect | | |
| Antimicrobial | Lactoferrin | Inhibits growth of | | | Tomita 1994- |
| effect | Casocidin I (casein α | pathogenic strains | | | Zucht 1995 |
| | S₁)-165-203 | | | | |
| | Casein αS₁B fragment | Inhibits growth of | Mouse, Sheep: effective | | Lahov 1996 |
| | (1-23 N terminal) = | pathogenic strains | as an IM injection against | | |
| | isacridin | | Staphylococcus aureus | | |
| | Human β casein | | Mouse: protective effect as | | Migliore - |
| | fragment | | an IV injection against | | Samour 1989 |
| | | | K. pneumoniae | | |
| Immunomodulating | Fragments of bovine α | Proliferation of human | | | Kayser 1996 |
| effect | lactalbumin and bovine | lymphocyte activity (PBL) | | | |
| | κ casein | by Con A | | | |
| | Synthetic β casokinin 10 | Proliferation or suppression | | | Kayser 1996 |
| | and β casomorphin 7 | of PBL depending on | | | |
| | | concentration | | | |
| | Human β casein 54-59 | Simulation of phagocytosis | | | Parker 1984 |
| | α lactalbumin 51-53 | of sheep red blood cells by | | | |
| | | mouse peritoneal | | | |
| | | macrophages | | | |
| | Bovine β casein | Stimulation of mouse | No in vivo protection | | Migliore - |
| | Casein 191-193 | peritoneal macrophages | | | Samour 1989 |
| | Casein 63-68 | | | | |
| | Bovine κ casein | Inhibition of lymphocyte B | | | Otani 1992, |
| | Casein-macropeptides | proliferation of Peyer | | | 1995 |
| | (106-169) | platelets in mice and | | | |
| | | rabbits | | | |
| Antithrombotic | Bovine | | | CGP isolated in | Chabance |
| effect | caseinoglycopeptide | | | the plasma of | 1995 |
| | (bCGP) | | | neonates after | |
| | Human | | | ingestion of | |
| | caseinoglycopeptide | | | formula milk | |
| | (hCGP) | | | or mother's milk | |
| | Peptide 106-116 of | Inhibition of platelet | | | Jollès |
| | bovine κ casein | aggregation | | | 1986 |
| | Human lactotransferrin | Inhibition of platelet | | | Raha |
| | tetrapeptide (39-42) | aggregation | | | 1988 |
| | | | Rats and guinea-pigs with | | Drouet 1990 |
| | | | experimental arterial | | |
| | | | thrombosis: after IV | | |
| | | | injection, antithrombotic | | |
| | | | activity | | |
| Antihypertensive | Enzyme hydrolysates | Inhibition of ACE | | | Mullaly |
| effect | of β lactoglobulin | | | | 1997 |
| | and α lactalbumin | | | | |
| | Synthetic fragments | Inhibition of ACE | Rats receiving | | Kohmura |
| | of human β casein | | angiotensin I: after | | 1989 |
| | | | IV injection, return | | |
| | | | to initial blood | | |
| | | | pressure level | | |
| | Peptides of milk | | Hypertensive rats: | | Masuda |
| | fermented by | | ingestion of 10 | | 1996 |
| | L. helveticus and | | mL of fermented | | |
| | S. cerevisiae | | milk/kg of body | | |
| | | | weight, peptides | | |
| | | | found in the | | |
| | | | aorta with ACE inhibition | | |
| | Peptides resulting | | Hypertensive rats: | | Yamamoto |
| | from milk fermented | | after ingestion, | | 1994 |
| | by L. helveticus | | reduction in blood | | |
| | | | pressure | | |
| | Peptides resulting | | Hypertensive rats: | | Nakamura |
| | from fermentation | | after ingestion, | | 1995 |
| | of milk by L. helveticus + | | reduction in blood | | |
| | S. cerevisiae Val-Pro-Pro | | pressure | | |
| | (VPP)/II-Pro-Pro (IPP) | | Normal rats: no effect | | |
| | | | | Humans with | Hata 1996 |
| | | | | hypertension (36 | |
| | | | | patients): after | |
| | | | | 8 weeks of | |
| | | | | ingestion of | |
| | | | | 95 mL/day, | |
| | | | | reduction in blood | |
| | | | | pressure | |
| Opiate | β casomorphins | | Rats: after intracarotid | | Ermisch |
| effects | | | injection, accumulation | | 1983 |
| | | | of β casomorphins in | | |
| | | | the area without a | | |
| | | | haematoencephalopathic | | |
| | | | barrier | | |
| | | | Newborn calves: after | | Umbach |
| | | | their first cow's milk | | 1985 |
| | | | meal, β casomorphins | | |
| | | | in the blood | | |
| | | | Piglets: after ingestion | | Meisel 1986 |
| | | | of bovine casein, β | | |
| | | | casomorphin isolated | | |
| | | | in the duodenal chyma | | |
| | | | Puppies: after | | Singh 1989 |
| | | | ingestion of mother | | |
| | | | milk, occurrence of | | |
| | | | β casomorphins in | | |
| | | | the blood | | |
| | | | | Humans: after | Svedberg |
| | | | | ingestion of cow's | 1985 |
| | | | | milk, presence of β | |
| | | | | casomorphins in the | |
| | | | | intestines | |
| | | | | but no blood in adults | Teschemacher |
| | | | | | 1986 |
| | Synthetic human | Opiate effect on | | | Yoshikawa |
| | β casein | the ileum isolated | | | 1986 |
| | peptides | from guinea-pigs, | | | |
| | | cancelled by | | | |
| | | naloxone | | | |
| | Human and | Opiate antagonist | | | Chiba 1989 |
| | bovine casoxins | effects on ileum | | | |
| | (κ casein) | muscle isolated | | | |
| | | from guinea-pigs | | | |

Advantageously, the food products according to the invention are functional foodstuff.

As briefly mentioned above, the term “functional foodstuff” refers to a foodstuff that advantageously affects one or more target function in the organism independently of its nutritional effects. It can thus result in an improvement in health and/or well-being and/or reduce the risk of occurrence of disease in the consumer who ingests normal quantities of said product. As an example of the effects of a “functional foodstuff”, we can cite the follow effects: anti-cancer, immunostimulating, promoting bone health, anti-stress, opiate, anti-hypertensive, improved calcium bioavailability and antimicrobial effects.

Such functional foodstuff can be for human and/or animal use.

According to one embodiment, the food products covered by this invention are fresh products.

According to another embodiment, the food products covered by this invention are chosen in particular from among: drinks, yoghurts, cream desserts, fermented milks or juices.

The culturing of the strains of the invention, pure or associated with other strains, can also be used probiotically in human or animal foodstuff or even as a lactic ferment, for example in the process described above.

Moreover, this invention also relates to the use of at least one natural strain of AMI-deficient S. thermophilus with reduced post-acidification and/or several natural variants of the latter, also AMI-deficient with reduced post-acidification and/or their biologically pure cultures and fractions of cultures to prepare fermented food products whose organoleptic properties are preserved during storage.

This invention is illustrated by the following figures:

FIG. 1: graphs of the kinetics of acidification of the AMI-functional mother strain I-3299 and AMI-deficient strain I-3301.

Milk: milk without casein hydrolysate; milk+N3 0.2 g/L; milk to which is added 0.2 g of ingredient N3 (ref. 211693, Difco, USA) per litre of milk.

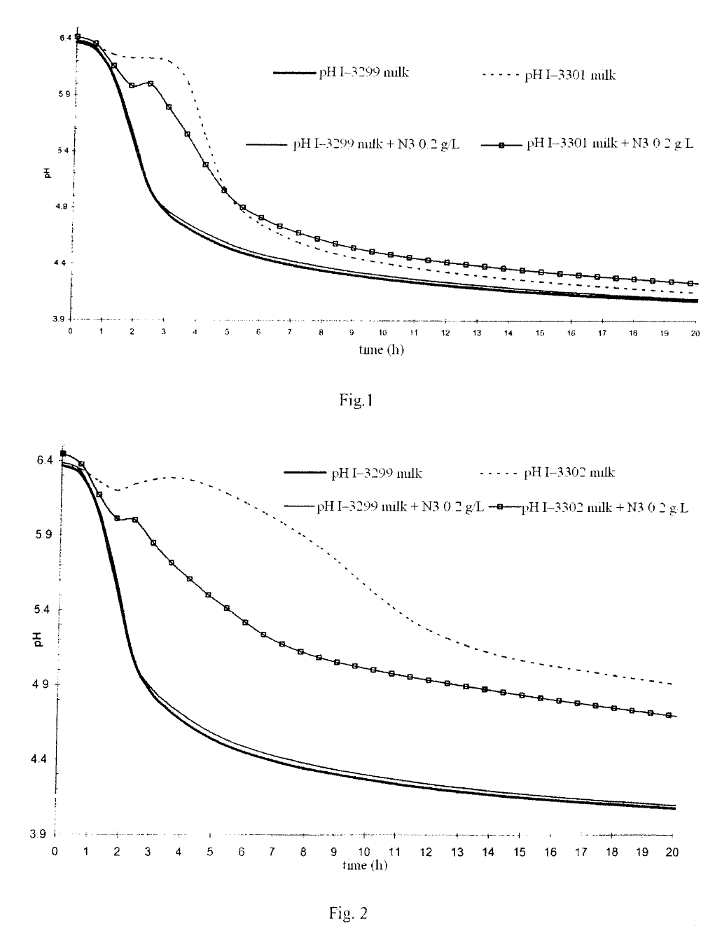

FIG. 2: graphs of the kinetics of acidification of the AMI-functional mother strain I-3299 and AMI-deficient strain I-3302.

Milk: milk without casein hydrolysate; milk+N3 0.2 g/L; milk to which is added 0.2 g of ingredient N3 per litre of milk.

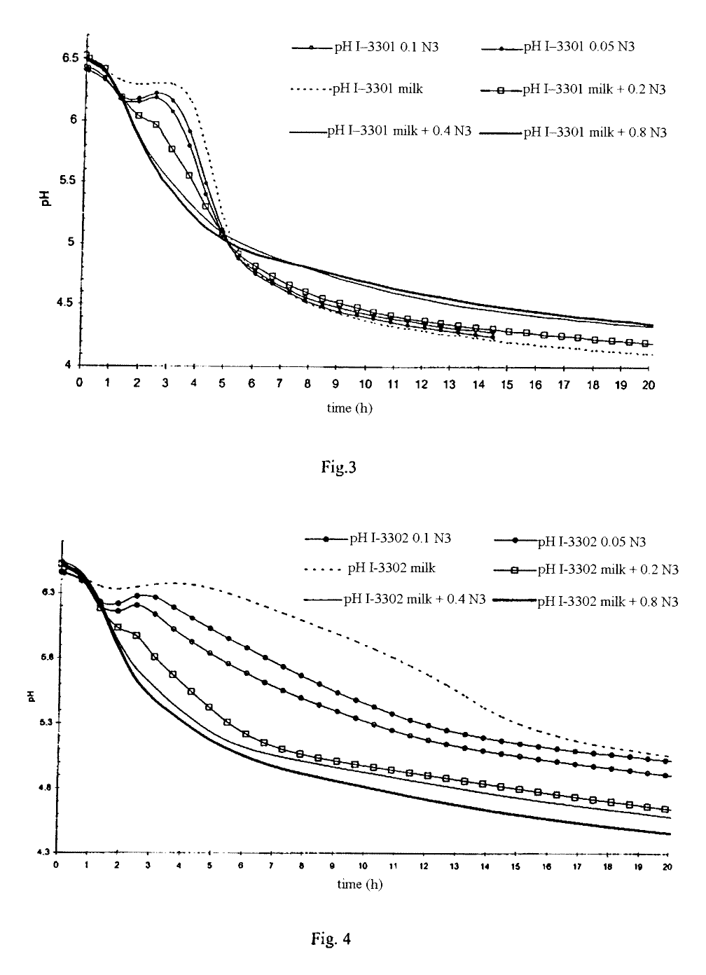

FIG. 3: graphs of the kinetics of acidification of the AMI-deficient strain I-3301 in the presence of varying amounts of N3.

Milk: milk without casein hydrolysate; milk+0.05 N3: milk to which is added 0.05 g of ingredient N3 per litre of milk; milk+0.1 N3: milk to which is added 0.1 g of ingredient N3 per litre of milk; milk+0.2 N3: milk to which is added 0.2 g of ingredient N3 per litre of milk; milk+0.4 N3: milk to which is added 0.4 g of ingredient N3 per litre of milk; milk+0.8 N3: milk to which is added 0.8 g of ingredient N3 per litre of milk.

FIG. 4: graphs of the kinetics of acidification of the AMI-deficient strain I-3302 in the presence of varying amounts of N3.

Milk: milk without casein hydrolysate; milk+0.05 N3: milk to which is added 0.05 g of ingredient N3 per litre of milk; milk+0.1 N3: milk to which is added 0.1 g of ingredient N3 per litre of milk; milk+0.2 N3: milk to which is added 0.2 g of ingredient N3 per litre of milk; milk+0.4 N3: milk to which is added 0.4 g of ingredient N3 per litre of milk; milk+0.8 N3: milk to which is added 0.8 g of ingredient N3 per litre of milk.

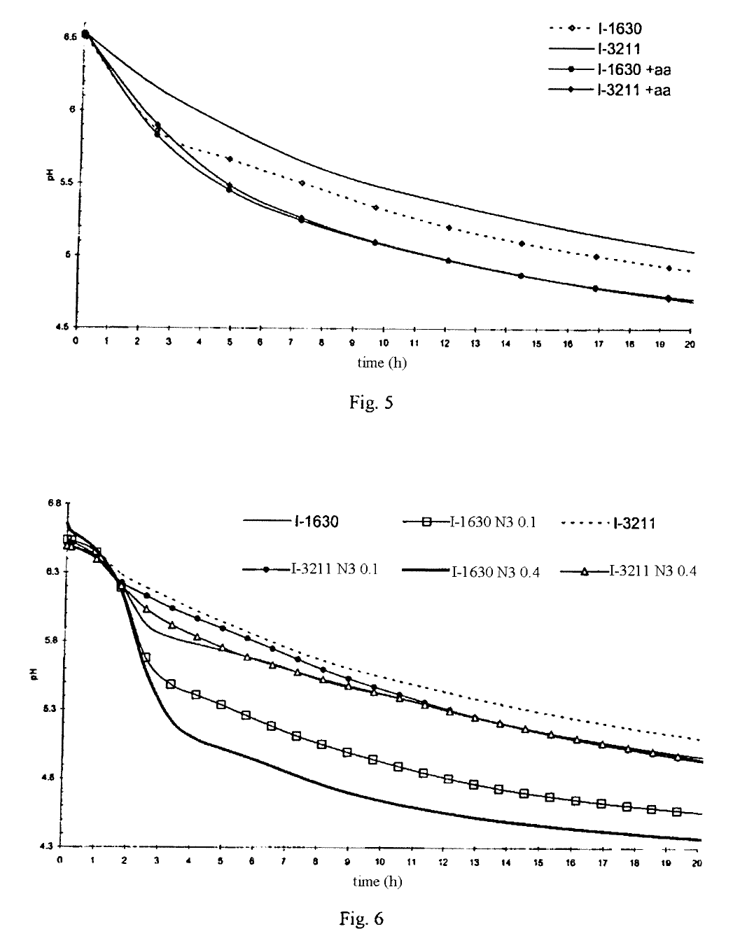

FIG. 5: graphs of the kinetics of acidification obtained from milk fermentation after addition of amino acids with AMI-functional mother strain I-1630 (registered with CNCM on Oct. 24, 1995) and AMI-deficient strain I-3211.

Name of strain without other details: milk fermentation; name of strain+aa: fermentation carried out with the addition of 20 amino acids (corresponding to 1/40^{th} of the quantity specified for the chemically defined medium adapted to S. thermophilus (Letort et al., 2001).

FIG. 6: graphs for kinetics obtained with milk to which ingredient N3 is added.

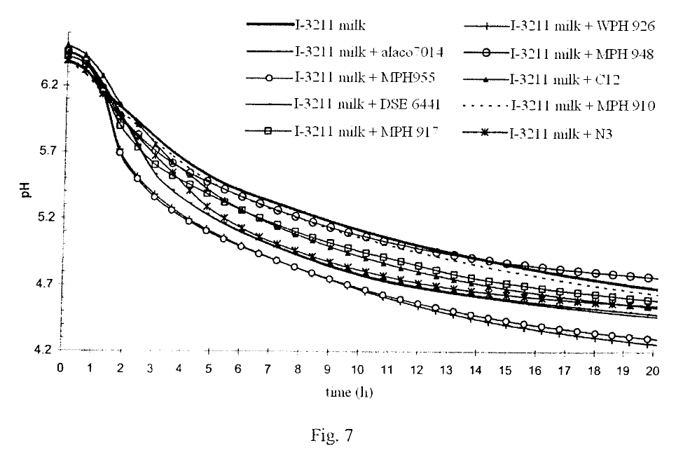

FIG. 7: graphs for acidification kinetics obtained with various ingredient mixtures.

Other features and advantages of this invention will become apparent on reading the examples shown in the experimental section below and given for the purpose of illustration only.

### Experimental Section

I. Materials and Methods

I.1. Obtaining AMI-Deficient Variants:

A) Evaluation of the Biodiversity of Resistance to Aminopterine in S. thermophilus

Initially, the sensitivity of S. thermophilus strains to aminopterine was verified. To do this, 200 μg of aminopterine were deposited on a disc placed in a Petri dish containing Elliker medium on which 100 μL of bacterial suspension were deposited. If the strain is sensitive, an inhibition diameter appears around the disc after incubation overnight. Mother strains I-1630 (registered with CNCM on Oct. 24, 1995) and I-3299 (registered with CNCM on Sep. 16, 2004) were selected as they possess interesting textural properties. Strain I-1630 has no wall protease whereas strain I-3299 does. These two strains therefore have very different growth characteristics.

TABLE 3

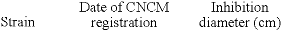

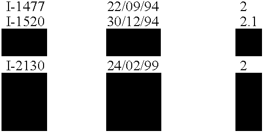

Different sensitivities were observed depending on the strain in question.

In table 3, the AMI-deficient variants obtained are give in bold in the shaded boxes, where necessary with their respective mother strains.

Resistance to aminopterine is greater in the variants compared to the corresponding mother strain.

The wild strain I-2774 shows natural resistance to aminopterine seen by its deficiency in an AMI transport system.

b) Obtaining AMI-Deficient Variants

The protocols for selecting deficient variants for peptide transport using a toxic peptide analogue have already been described (Higgins and Gibson, 1986).

In brief, an aminopterine range from 0 to 200 μg/mL was made up with a chemically defined medium (MCDaa) adapted to S. thermophilus growth and containing 20 free amino acids as a source of nitrogen.

All the tubes in the range were seeded at 1% using a preculture made up in MCDaa. After incubation for three days at 37° C. 100 μL of each culture tube was used to make up a bacterial carpet on an Elliker agar medium. A disc on which 200 μg of aminopterine was deposited placed at the centre of the dish. The dishes were then incubated for three days at 37° C. under anaerobic conditions.

The presence of clones in the inhibition zones was then observed and around 20 clones were removed and used to seed 5 mL of Elliker medium.

The clones were divided up in this medium and gave rise to bacterial suspension after 16 hours of incubation at 37° C. This led to a carpet being formed on the surface of the Elliker medium onto which a disc soaked in 200 μg of aminopterine was deposited.

All the clones obtained were then tested by fermentation in milk. The kinetics of acidification provided information about the milk-acidifying capacity of these variants.

I.2. Preparation of Milk:

Milk was reconstituted with 120 g of skimmed powder in 930 mL of water. After 30 minutes of hydration, the milk was pasteurised for 30 minutes at 95° C.

Ingredient N3 was added in the form of a 10% solution in water then sterilised by 0.22 μm filtration.

The other ingredients tested were added to the milk prior to pasteurisation.

The amino acid solution was made up in water then sterilized by 0.22 μm filtration.

I.3. Preparation of Ferments for Acidification Follow-Up:

A preculture was made up in sterilised milk with a yeast autolysate seeded at 1%, incubated for 18 hours at 42° C. Next the ferment was made up in 100 mL of sterilised milk with autolysate incubated at 42° C. and stopped when acidity reached 80° D by placing in ice water. The ferment was stored at 4° C. until the commencement of acidification follow-up.

250 mL of milk were seeded at 1% with the S. thermophilus ferment in order to launch the acidification follow-up.

I.4. Materials

N3: protease peptone No 3, ref. R211693, Difco, USA (casein hydrolysate containing peptides and, mainly, free amino acids).

Milk powder: Milex 240, Arla Food Ingredients.

Yeast autolysate: yeast extract, ref. AEB171109, AES Laboratories.

CINAC for acidification follow-up: Automatic system for the measurement of acidifying activity developed by G. Corrieu, LGMPA, YSEBAERT brand.

Alaco 7014: lactoserum protein hydrolysate, NZMP Gmbh, Siemens Strasse, 6-14 D-25463, Rellingen, Germany.

MPH 955: casein hydrolysate, NZMP Gmbh, Siemens Strasse, 6-14 D-25463, Rellingen, Germany.

DSE 6441: lactoserum protein hydrolysate, NZMP Gmbh, Siemens Strasse, 6-14 D-25463, Rellingen, Germany.

MPH 917: casein hydrolysate, NZMP Gmbh, Siemens Strasse, 6-14 D-25463, Rellingen, Germany.

WPH 926: lactoserum protein hdyrolysate, NZMP Gmbh, Siemens Strasse, 6-14 D-25463, Rellingen, Germany.

MPH 948: casein hdyrolysate, NZMP Gmbh, Siemens Strasse, 6-14 D-25463, Rellingen, Germany.

C 12: milk protein hydrolysate, DMV International NCB Laan 80, PO BOX 13 5460, Ba Zeghel, Netherlands.

MPH 910: casein hdyrolysate, NZMP Gmbh, Siemens Strasse, 6-14 D-25463, Rellingen, Germany.

II. Results

II.1. Acidification Kinetics of AMI-Functional Mother Strain I-3299 and Derivative AMI-Deficient Strains in the Presence or not of Ingredient N3

It can be seen from FIGS. 1 and 2 that the AMI-functional mother strain I-3299 has the same kinetics in the presence and absence of ingredient N3 in the milk.

On the other hand, the AMI-deficient strains have different kinetics.

II.2. Acidification Kinetics of Varying Amounts of Ingredient N3, AMI-Deficient Strain I-3301

Strain I-3301 shows kinetics with a shorter latency in the presence of ingredient N3. In addition, pH at the end of fermentation is higher the more ingredient N3 there is in the medium.

II.3. Acidification Kinetics of Varying Amounts of Ingredient N3, AMI-Deficient Strain I-3302

The acidification kinetics are faster the more ingredient N3 there is in the medium. In addition, pH at the end of fermentation is lower the more ingredient N3 there is in the medium.

II.4. Post-Acidification of the Pure Strain (Compared to Mother Strain I-3299)

TABLE 4

| Times (days) | PH I-3302 | PH I-3299 |
|---|---|---|
| PH at end of incubation | 4.70 | 4.70 |
| PH at D 28 (10° C.) | 4.41 | 4.14 |
| δpH | 0.29 | 0.56 |

The AMI-deficient strain makes it possible to have a pH difference (delta) of 0.29 between the pH at the end of incubation and the pH of the product at the end of its shelf life whereas the mother strain gives rise to delta pH 0.56. The advantage of using the AMI-deficient strains is the 0.27 unit pH if we compare the δpH under these conditions.

II.5. Acidification Kinetics Obtained with Fermentation of Milk after Addition of Amino Acids with the AMI-Functional Mother Strain I-1630 (Registered with CNCM on Sep. 24, 1995) and the Derivative AMI-Deficient Strain I-3211

The stimulation generated by the addition of free amino acids to the milk is the same for the mother strain and the AMI-deficient strain. This result is logical insofar as the free amino acids represent the only available source of nitrogen for the two strains under these conditions.

II.6. Kinetics Obtained with Milk+Ingredient N3

The AMI-functional mother strain I-1630 (registered with CNCM on Oct. 24, 1995) is clearly stimulated by the addition of ingredient N3 to milk whereas the derivative AMI-deficient strain is not. The N3 peptide hydrolysate can be used by the mother strain but not by the AMI-deficient strain.

II.7. Acidification Kinetics with Various Ingredients with the AMI-Deficient Strain I-3211

The MPH955 and WPH926 hydrolysates lead to better stimulation of fermentation of the AMI-deficient strain in milk. They are also the two hydrolysates with the highest content in small peptide and free amino acids.

### REFERENCES

Garault, P., et al., (2002) Journal of Biological Chemistry 277: 32-39
Letort et al., (2001) J. Appl. Microbiol. 91:1023-1029
Letort, C, et al., (2002) Applied and Environmental Microbiology 68:3162-3165
Spinnier H. E., et al. Automatic method to quantify starter activity based on pH measurement” J. of Dairy Research, 56 (1989) 755-764); de Roissart H. et Luquet F. M. Edition Lorica ISBN: 2-9507477-0-1
Kayser et al., (1996) FEBS Letters 383, 18-20
Hata Y. et al., (1996) Am. J. Clin. Nutr. 64, 767-71
Nakamura Y. et al., (1995) J. Dairy Sci. 78, 1253-7
Migliore-Samour D. et al., (1988) Experimentia 44, 188-93
Defilippi C. et al., (1995) Nutr. 11, 751-4
Tome D. et al., (1987) Am. J. Physiol. 253, G737-44
Tome D. et al., (1988) Reprod. Nutri. Dévelop. 28, 909-18
Ben Mansour A. et al., (1988) Pediatr. Res. 24, 751-5
Mahé S. et al., (1989) Reprod. Nutri. Dévelop. 29, 725-32
Schusdziarra V. et al., (1983) Diabetologia 24, 113-6
Yvon M. et al., (1994) Reprod. Nutri. Dévelop. 34, 527-37
Zucht H. D., et al., (1995) FEBS Letters 372, 185-8
Tomita M. et al., (1994) Acta Paed. Jap. 36, 585-91
Lahov E. et al., (1996) Food Chem. Toxic. 34, 131-145
Migliore-Samour D. et al., (1989) Int. Dairy Res. 56, 357-62
Jolies P. et al., (1986) Europ. J. Biochem. 158, 379-82
Raha S. et al., (1988) Blood 772, 172-8
Chabance B. et al., (1995) Brit. J. Nut. 73, 582-90
Kohmura M. et al., (1989) Agric. Biol. Chem. 53, 2107-14
Masuda O. et al., (1996) J. Nutr. 126, 3063-8
Yamamoto N. et al., (1994) Biosci. Biotech. Biochem. 58, 776-8
Ermisch A. et al., (1983) J. Neurochem. 41, 1229
Umbach M. et al., (1985) Regul. Pept. 12, 223-30
Singh M., et al., (1989) Pediatr. Res. 26, 34-8
Svedberg J. et al., (1985) Peptides 6, 825-30
Teschemacher H., et al., (1986) J. Dairy Res. 53, 135-8
Yoshikawa M. et al., (1986) Agric. Biol. Chem. 50, 2419-21
Chiba H. et al., (1989) J. Dairy Sci. 72, 363
Beucher S. et al., (1994) J. Nutr. Biochem. 5, 578-84
Parker F. et al., (1984) Eur. J. Biochem. 45, 677-82
Otani H. et al., (1992) Milchwiss. 47, 512-5
Otani H. et al., (1995) J. Dairy Res. 62, 339-48
Drouet et al., (1990) Nouv. Rev. Fr. Hermatol. 32, 59-62
MuIIaIy M. et al., (1997) Int. Dairy J. 7, 299-303
Meisel H. et al., (1986) FEBS Letters 196, 223-7
Danone World Newsletter No 17 (September 1998)
Higgins CF. et Gibson M. M., (1986) Peptide transport in bacteria.
Methods in Enzymology 125, 365-377

1. An isolated AMI-deficient Streptococcus thermophilus strain with reduced post-acidification and its natural AMI-deficient variants having similar milk-acidification characteristics, as well as their biologically pure cultures and culture fractions, with the exclusion of the strain registered with CNCM on Jan. 24, 2002 under number I-2774.

2. The strain according to claim 1, wherein said strain is resistant to a toxic oligopeptide analogue transported by the AMI system.

3. The strain according to claim 2, wherein said toxic oligopeptide analogue is aminopterine.

4. The strain according to claim 3, wherein the inhibition diameter observed in the presence of a paper disc soaked in 200 μg of aminopterine is less than or equal to about 2 cm, preferably less than or equal to about 1.8 cm, in a dish seeded with 100 μl of saturated bacterial suspension.

5. The strain according to claim 1, wherein said strain is chosen from the group consisting of:
the strain registered with CNCM on Oct. 5, 2004 under number I-3211;
the strain registered with CNCM on Sep. 16, 2004 under number I-3301; and
the strain registered with CNCM on Sep. 16, 2004 under number I-3302.

6. The strain according to claim 1, wherein said natural AMI-deficient variants are obtained by mutation and selection of their acidifying properties.

7. A method for the preparation of a fermented food product in which the substrate is fermented with at least one live strain according to claim 1.

8. The method according to claim 7, wherein fermentation is carried out in the presence of at least one other live bacterial strain.

9. The method according to claim 8, wherein said other live bacterial strain is a live lactic bacteria.

10. The method according to claim 9, wherein said live lactic bacteria is chosen from the group consisting of Streptococcus spp.; Lactobacillus spp., namely L. bulgaricus, L. acidophilus and L. casei; Lactococcus spp. and Bifidobacterium spp.

11. The method according to claim 7, wherein said substrate is chosen from the group consisting of dairy substrates, namely milk, and substrates based on vegetable raw ingredients such as fruits, cereals and soya.

12. The method according to claim 11, wherein said substrate contains solid elements.

13. The method according to claim 12, wherein said solid elements are chosen from the group consisting of fruits, chocolate-containing products and cereals.

14. A fermented food product containing at least one live strain according to claim 1.

15. The fermented food product obtainable by a method according to claim 7.

16. The fermented food product according to claim 14, wherein it is a dairy product or a vegetable product.

17. The fermented food product according to claim 14, wherein it contains at least one bioactive food ingredient.

18. The fermented food product according to claim 17, wherein said bioactive food product is chosen from the group consisting of proteins, peptides and their analogues and derivatives.

19. The fermented food product according to claim 17, wherein it is a functional food.

20. The fermented food product according to claim 14, wherein it is a fresh product.

21. The fermented food product according to claim 14, wherein it is chosen from the group consisting of drinks; yogurts and cream desserts; fermented milks and juices.

22. The fermented food product according to claim 14, wherein its organoleptic properties are preserved during storage.

23. (canceled)

24. (canceled)

25. (canceled)

26. A method for obtaining a lactic ferment, comprising at least one strain according to claim 1.

27. A method for obtaining a probiotic in human or animal foodstuffs comprising at least one strain according to claim 1.
